(19) United States
(12) Patent Application Publication
(10) Pub. No.: US 2022/0015872 A1
(43) Pub. Date: Jan. 20, 2022

(54) APPARATUS AND METHOD FOR REHABILITATION OF GUM ARCHITECTURE

(71) Applicant: FLAVIO SADAMO MICIMA, CURITIBA- Parana (BR)

(72) Inventors: FLAVIO SADAMO MICIMA, CURITIBA (BR); FABIANO KENDY MICIMA, CURITIBA (BR); FELIPE UEIKI MICIMA, CURITIBA (BR)

(21) Appl. No.: 16/928,908

(22) Filed: Jul. 14, 2020

(51) Int. Cl. A61C 8/02; A61C 8/00; A61K 9/00; A61K 47/34; A61P 1/02

(52) U.S. Cl. CPC A61C 8/0006; A61C 8/0093; A61C 8/004; A61C 2201/00; A61K 47/34; A61P 1/02; A61K 9/0024

(57) ABSTRACT

The invention relates to dental field, more specifically in the field of periodontics, and describes the construction and working of a modular gum architecture device made of inert, biocompatible, bone-integrable and/or absorbable material to replace in part or totally the lost alveolar bone and restore the anatomy of the gum around the tooth or implant. The device may recreate the region of the interdental papilla or may fill in empty spaces caused by protocol-type prostheses (fixed dentures). It can also serve as support for teeth already softened or softening, for implants already installed or even for implants to be installed.

### FIELD OF THE INVENTION

The present invention is in the dental field, more specifically in the field of periodontics, and describes the construction and working of a modular gum architecture device made of inert, biocompatible, bone-integrable and/or absorbable material for replace in part or totally the lost alveolar bone and method to restore the anatomy of the gum around the tooth or implant. The device may recreate the region of the interdental papilla or refill empty spaces caused by protocol-type prostheses (fixed dentures). It can also serve as support for teeth already softened or softening, for implants already installed or even for implants to be installed.

Gum architecture is the anatomy of the gum that surrounds the teeth which is responsible for the natural aspect and where the teeth are inserted in the gum.

Loss of gum anatomy are caused by several factors, including trauma that ends up damaging the alveolar bone, compromising the gum support structure. Another factor is the bacteria accumulated in plaques that can cause periodontal disease, which has the ability to cause inflammation by attacking the gum connective tissue, periodontal ligaments and bone tissues, compromising the gum anatomy around the teeth, one of which is the gum papilla.

The papilla is a dense connective tissue covered by pyramid-shaped epithelium and aims to serve as a biological barrier deflecting food and preventing inflammation due to the accumulation of dirt, it also serves as aesthetics, avoiding a dark space between the teeth.

Another problem that the present patent solves is the space caused by the protocol-type prosthesis system, space that occurs between the patient's natural gum and the artificial gum (flange) of the prosthesis used for cleaning. Spaces interfere with diction, accumulate dirt that causes perimplantitis and have bad aesthetic aspects, which can cause low self-esteem of the patient or cause inflammation and/or infections leading to loss of implants.

The patents Brazil PI 0603264-8 and U.S. Pat. No. 6,213,774 B1 disclose a dental pin fixed for rehabilitation of the interdental bone crest, serving as support for the soft tissue punctually. Unlike the present invention disclosed here, it is not a pin but a structure, where it replaces the lost alveolar bone around the natural teeth or implants, having the ability to recover all or part of the gum anatomy.

The U.S. Pat. Nos. 7,296,997 and 7,934,928, although they are not pins like the patents mentioned above, they are still devices that solve only the region of the interdental papilla and for the same reasons already mentioned, they do not solve problems of the structure around the teeth, being exclusive for papilla recovery (interdental region).

The patent WO 2013121451 presents a device in which prevents the resorption of the interdental bone peak, that is, there is a need for the interdental bone peak to exist, therefore it does not solve the cases of total loss of the interdental bone peak and/or below of this peak.

The U.S. Pat. Nos. 5,499,918 and 4,531,916 disclose artifacts of biocompatible material that have a support structure for the gum tissue on the implant structure, thus, the need to have an implant, while the present patent is not limited to the dental implant, since it can be installed around natural teeth and serve as support for implants.

The U.S. Pat. No. 6,911,046 provides a biocompatible form that refers to a situation of reconstructing the missing bone in the oral cavity of an exclusively edentulous patient, contrary to the present patent disclosed here which has the function of restoring the alveolar bone of both natural teeth and implants, unlike U.S. Pat. No. 6,911,046, which has a unique shape and it is useful for use only by toothless individuals.

The U.S. Pat. No. 5,769,637 discloses a prosthetic device to increase the volume of an alveolar bone and promote guided regeneration of bone tissue but does not restore the gum architecture with papilla and the emerging profile of the tooth crown.

The patents WO 2017/083922 and WO 02/19921 disclose a base for implants, however, they only cover cases of artificial teeth while the patent disclosed here has a solution for natural teeth and restores the gum architecture through the anatomy of the piece.

Unlike the state of the art that presents only partial solutions, the invention disclosed through this document presents solutions for restoration of the gum architecture due to the absence of alveolar bone lost partially or in full caused by periodontal diseases, trauma or any other reason in which occurs loss of alveolar bone. Another objective of the device is to solve the problem caused by the aesthetics of the prosthesis protocol, where it leaves an empty space.

### SUMMARY OF THE INVENTION

The present invention discloses an apparatus that consists of a structure to replace the total or partially lost alveolar bone around the teeth and/or implants. The device helps as a support structure for soft tissues, helps to support teeth and/or implants, works as a lip support and also performs a function as a storage compartment (framework) of biomaterial, through three-dimensional holes in the device wall, which allow protect biomaterials resulting in an acceleration of nutrition, vascularization and innervation, consequently an acceleration of bone regeneration.

The invention's objectives are to replace the function of the alveolar bone, to restore the gum architecture around the teeth, including the interdental region, to restore the interdental papilla; to serve as mechanical support for teeth and implants; to cause a lip filling avoiding the appearance of withering; to fill in spaces between the natural and artificial gums in fixed dentures.

### DESCRIPTION OF THE DRAWINGS

The description which follows seeks to highlight the principle of the invention, without limiting to the designs of the components, having as reference the following illustrations listed below:

FIG. 1 shows a perspective view depicting an arrangement of a piece for total edentulous, highlighting the elevation for anatomy of the interdental papilla;

FIG. 2 shows a perspective view representing the appliance for a tooth;

FIG. 3 shows a perspective view representing a format that the device may have;

FIG. 4 shows an exploded perspective view representing modules that the device may have;

FIG. 5 shows a perspective view representing union of adjacent modules in an interdental region;

FIG. 6 shows a perspective view representing modules of an interdental region joined, highlighting elevation for interdental anatomy;

FIG. 7 shows a perspective view representing a configuration with a cervical flap;

FIG. 8 shows a perspective view showing a configuration for supporting implants;

FIG. 9 shows a sectional view for better representation of FIG. 7;

FIG. 10 shows a perspective view for representation of articulation in the interdental region;

FIG. 11 shows a perspective view for representation of articulation in the cervical region;

FIG. 12 shows a perspective view for representation of articulation in the vestibular region;

FIG. 13 shows a perspective view for representing a superstructure;

FIG. 14 shows a perspective view for fitting representation of the superstructure above the apparatus;

FIG. 15 shows a perspective view for representing the configuration of the superstructure in modules;

FIG. 16 shows a perspective view for representation of combination of superstructure;

FIG. 17 shows a view in perspective to represent holes in the device;

FIG. 18 shows a perspective view to represent the holes of the superstructure.

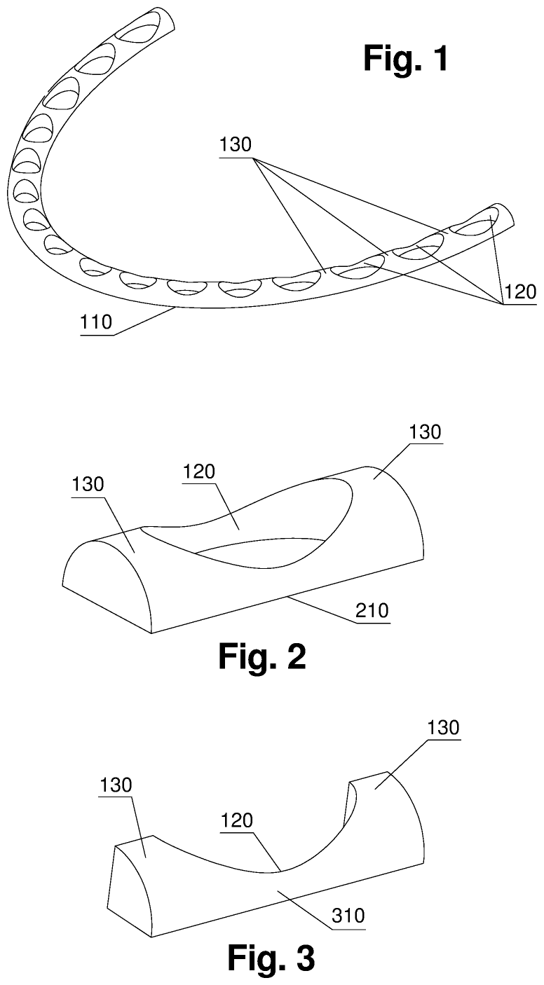

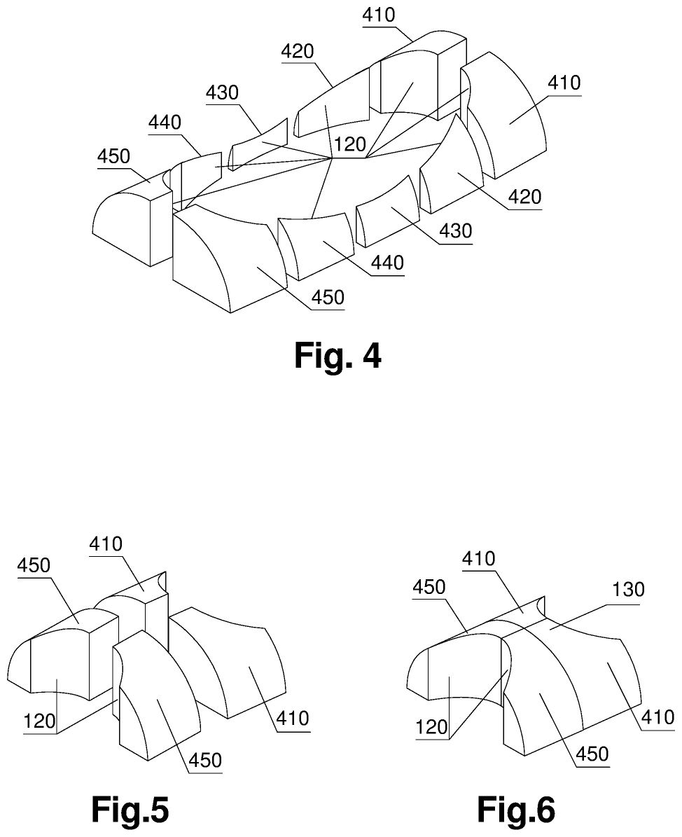

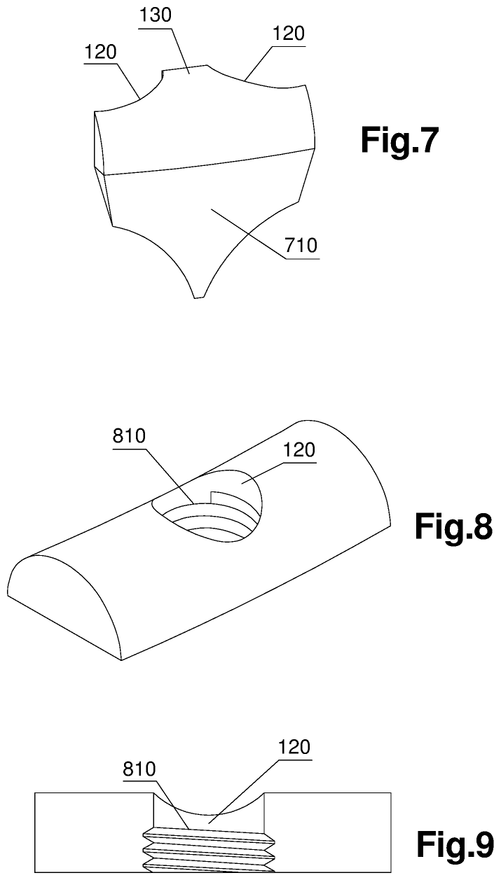

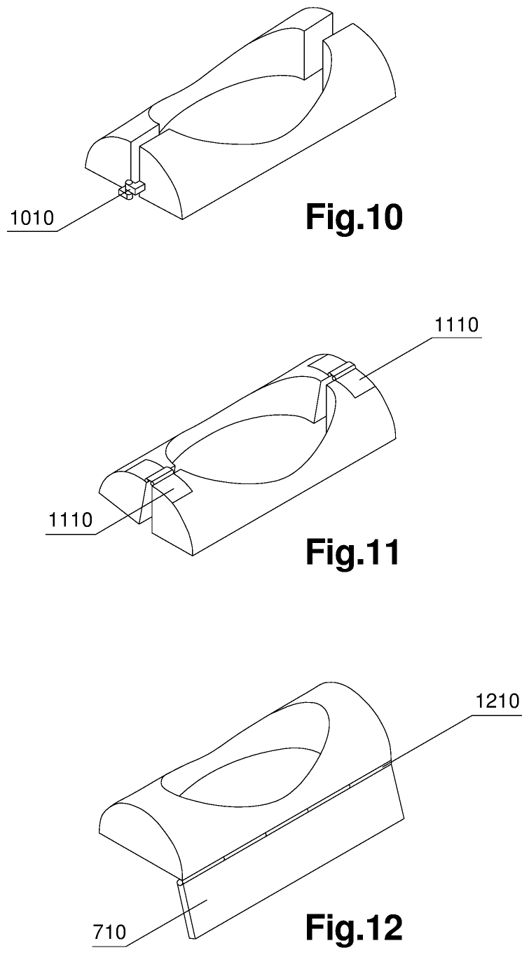

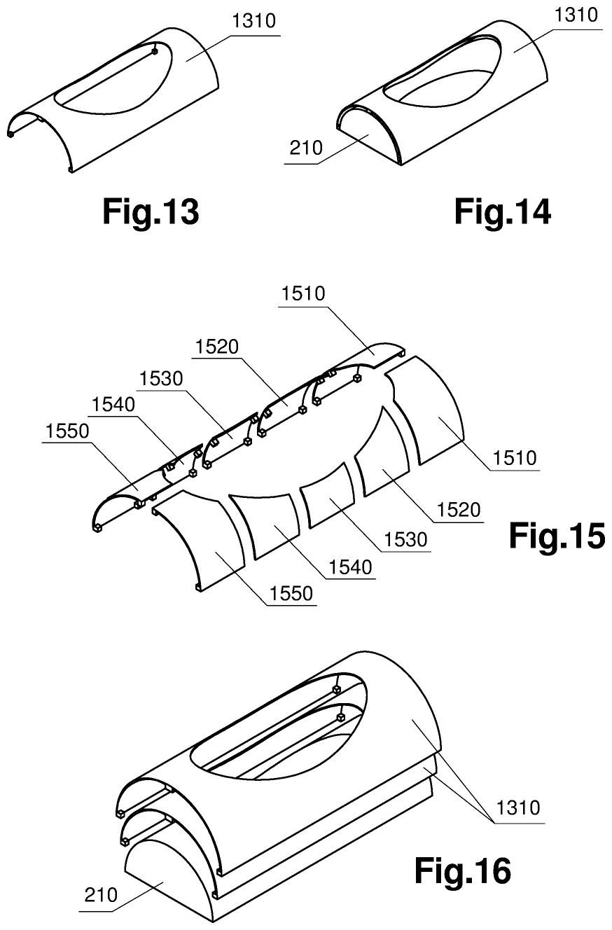

### DETAILED DESCRIPTION OF THE INVENTION

The device may have different formats that will depend on the need for the surgical procedure and the patient's situation. It can be:
A single piece for total edentulous (110);
A single piece with two or more holes for teeth/implants;
A single piece for only one tooth/implant (210);
The configuration (210) divided in half in a mesio-distal direction (310);
The configuration (310) be subdivided into small modules of buccal-lingual sense (410 to 450 and 410 to 450);
Or any combination of the items mentioned above.
From inert solid material, biocompatible, bone-integrable and in some cases resorbable.

The device can be installed in a single or combined way (FIGS. 5 and 6 exemplifies a type of case), and each part/module may vary in size and volume to solve cases of uneven generation.

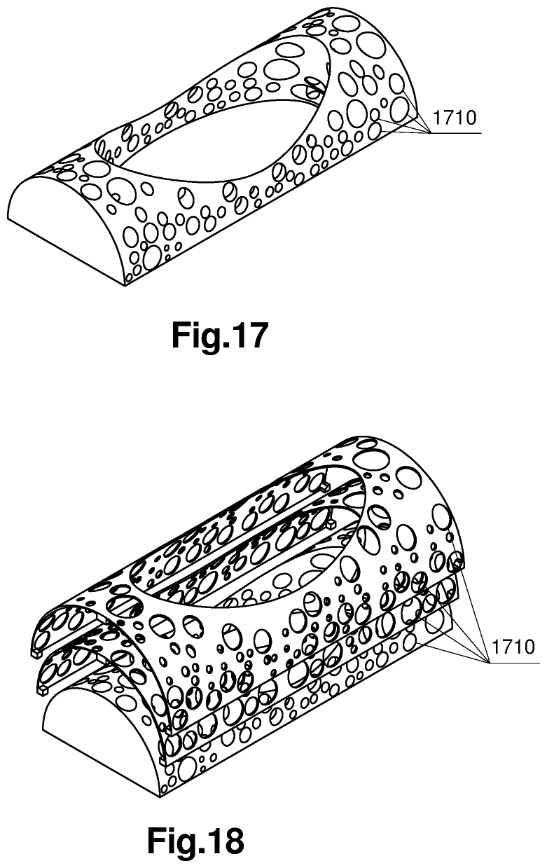

It must have a hole (120) for whole pieces or a curvature (120) resulting from the cutting of the modules to fit the natural tooth and/or implant, which may contain threads to assist in fixing implants (810). Although represented only in FIGS. 17 and 18, the device must have holes in all its configurations and accessories 1710 for irrigation, tissue fixation and/or for fixing the device to the bone through pins as well as serving as a storage compartment (frame) of biomaterial, allowing to protect it, resulting in an acceleration of nutrition, vascularization and innervation, consequently an acceleration of bone regeneration.

The device may have a lateral flap (130) in the interproximal region (between spaces of teeth and/or implants) that will serve as support for the connective tissue featuring in an interdental papilla structure. It may have a lower flap in the occlusal-cervical direction (710) to supply cases of bone lack in this direction.

It also has joints for better adaptation to the recipient bone bed, and these joints can be in the interdental (1010) and/or in the cervical (1110) and/or vestibular (1210) regions, combining with the occlusal-cervical flap (710).

There may also be a superstructure (1310) in the device to increase volume and height capable of supplying cases of loss of extreme bone. They provide modules (1510 to 1550 and 1510 to 1550) to fit with the device's modules (410 to 450 and 410 to 450). The superstructure (1310) can be combined, as shown in FIG. 16, to achieve greater height and volume.

The device can be contained in a biomaterial or material block that presents good biocompatibility and integrability, in a stratified way, to present better volume, compatibility and integrability characteristic. This combination of block and device will allow the professional to machine the block in order to solve the case.

The device must be installed below the gum and, according to each case, it can be attached to the bone or glued by pins in a juxtaposed, overlapping, semi-intraosseous or stratified way (recipient bone, particulate bone device or even between bone blocks).

In addition to the device, it is possible to rehabilitate the hard (bone) and soft (gum) tissues by a method of detaching the gum, similar to a bariatric surgery, and injecting biocompatible, bone integrable, inert, polymerizable, catalytic and catalytic and passable biomaterial modeling of the lost gum architecture, as well as having a rubber or silicone structure. Another method is to introduce a bladder-like structure in the form of a papilla below the gum where the tunnel detachment was made in the region surrounding the tooth or implants and the papilla. After the insertion of the bladder, it is filled with silicone or biomaterial capable of catalysis in order to take the shape of the necessary or desirable gum architecture.

Rehabilitation also occurs in case of more severe bone loss, including in the region of the basal bone (jaw and mandible) and in the region of the alveolar bone (bone below the alveolar bone that circulates natural teeth or implants).

The features of the device and method solve the problems of lack of alveolar bone as well as rehabilitate the gum architecture, giving a natural and pleasant aesthetic appearance to the user.

1- Apparatus for rehabilitation of gum architecture, comprising:
A solid, biocompatible, bone-integrable, inert and resorbable material, being defined by part (110),
dividable into modules (210, 310, 410 to 450) and the mirrored correspondents (410 to 450),
with an incision-cervical orifice (120) or curvature (120),
with or without a thread on the inside and holes (1710) in its structure.

2- Apparatus for rehabilitation of gum architecture, in accordance with claim 1, comprising:
flaps of occlusal-cervical (710) and mesio-distal (130) in the interproximal region

3- Apparatus for rehabilitation of gum architecture, according to claim 1, comprising joints (1010, 1110, 1210)

4- Apparatus for rehabilitation of gum architecture, according to claim 1, comprising a superstructure (1310) and its variant forms (1510 to 1550 and 1510 to 1550)

5- Apparatus for rehabilitation of gum architecture, according to claim 1, comprising a device contained in a biomaterial block

6- Method for rehabilitation of gum architecture, comprising the rehabilitation of hard (bone) and soft (gum) tissues by gum detachment and injection of biocompatible, integrable, inert, polymerizable bone material, catalytic able to obtain a modeling of the lost gum architecture, with a rubber or silicone structure

7- Method for rehabilitation of gum architecture, according to claim 6, comprising:
introducing a bladder-like structure in the form of a papilla below the gum after the tunnel detachment in the region surrounding the tooth or implants and papilla,
filling of this bladder with silicone or biomaterial liable to catalysis.
